(19) United States
(12) Patent Application Publication
(10) Pub. No.: US 2004/0097899 A1
(43) Pub. Date: May 20, 2004

(54) Disposable sani-panti

(76) Inventor: Webelene Bethea, Chicago, IL (US)

Correspondence Address: Webelene Bethea, 1322 W. 72nd Street, Chicago, IL, 60636, US

(21) Appl. No.: 10/295,130

(22) Filed: May 13, 2003

Related U.S. Application Data
(60) Provisional application No. 60/315,211, filed on Dec. 13, 2001.

(51) Int. Cl. A61F 13/15

(52) U.S. Cl. 604/393

(57) ABSTRACT

My invention is a sanitary hygiene disposable product wherein the sanitary napkin (pad) and the underwear (panty product) is all in one. The length of the napkin varies and is sew, manufactured or interwoven into the crouch area of the underwear (panti) garment. The pad extends in the crouch area and varies in length: equally long, for both the front/back area of the underwear or vice versa for the short napkin and available as short in front and long in back or vice versa.

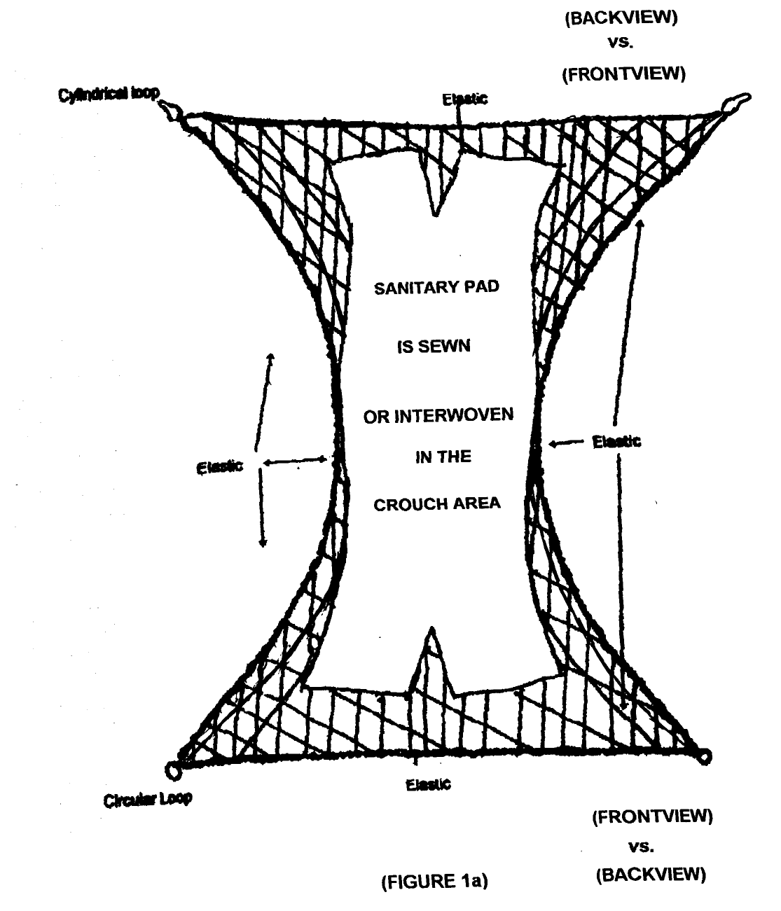

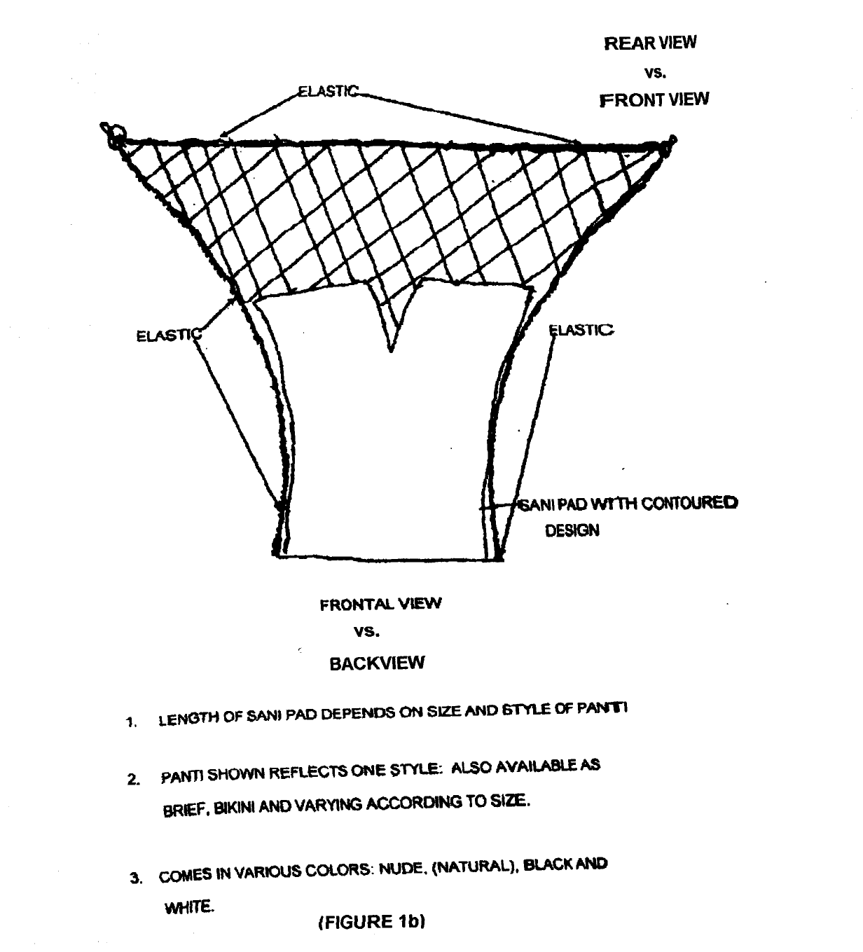

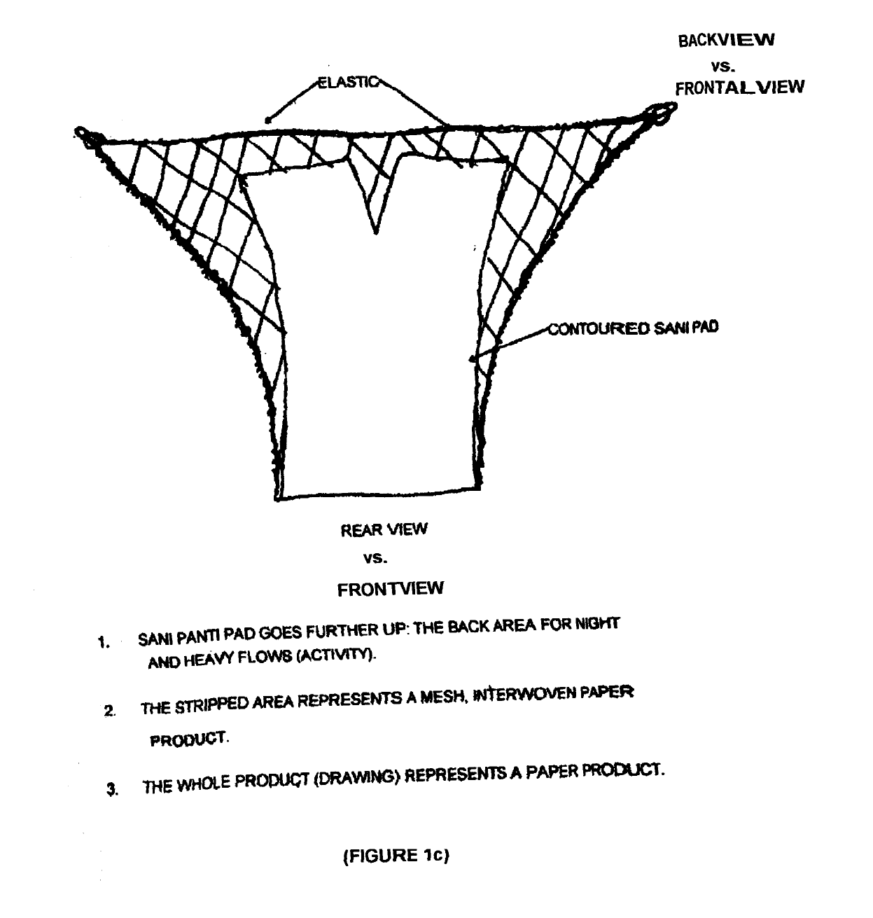

The design for the napkin is contoured for the pubic area: labia major wells for snug fit of the labia major and wedge shaped on the ends for elongation of the napkin. The napkin design is not limited and is adaptable in use.

The design styles are: bikini, brief, full, or thong, and the whole product (napkin/panti) are made of paper/cotton and or plastic.

What I claim as my invention is a hygiene paper product of a sanitary napkin and panty or underwear garment all in one: A disposable sanitary hygiene paper product for that time of the month: For pre and/or post menustrating or menuposal young girls, ladies and women. (Its use can be and is adaptable.)

The product is meant to be used as an all in one product: in lieu of separate underwear garment where the pad is secured or fastened to the crouch area of the panty. This product is meant to be used to avoid soiling of these undergarment or panties where in some circumstances the stain is impossible to remove thus ruining the continuing wear of the underwear (panty). So, this product is both a sanitary pad and a panty (panti) that can be folded up and disposed all in one, where in the panti part will also act as the wrapping.

The sanitary pad is made by being sewn, attached or woven into the crouch area of the panti.

Note: the use of the word sanitary napkin and pad are being used interchangably.

Shape:

The shape of the sanitary pad is wedge shaped and contoured to the shape of the lower pubic area. It fits snuggly because its' design is that of the imprint of the pubic area, i.e. (labia major). The wedge allows for the close fit of the pad due to the labia major wells. The contoured shape along with the wedge design allows for a snug-like fit of the labia major and acts as a cradle with double thickness allowing for longer tighter wear with a snugger fit: which is further enhanced by the elastic trimming on the top and leg portion of the panti. The overall panti is made of a mesh like paper, product or netting. The crouch area, is where the napkin is interwoven and extends/spreading outward due to the wedge design and depending upon the type of protection needed. The napkin (sanitary pad) can extend forwards, backwards equal or unequal in length again dependent upon the type of protection coverage needed. The panti is held in place around the buttock, leg, abdomen or lower area by fasteners: these fasteners will either be loop and circular, velcro like adhesives, toggle like style, or hooks.

The fasteners could be velcro like strips or hooks similar to that found on bras only plastic, because it is intended to be a disposable product or plastic hoops: one cylindrical to fit into a circular loop causing a latchlike attachment.

The Panti and Sanitary Pad are Presented as One Product

The interwoven or sewn pad extends beyond the crouch area and the panti is secured to the wearer by circular hooks, toggles, twists, ties or hooks that are there for adherence or secure fit:

The interwoven napkin is in varying lengths dependent upon the type of protection or condition: heavy, medium to lite days.

The napkin in the panti is expanded beyond the crouch area and contoured out slightly.

For Example:

The heavy is for (anemia) women who have heavy flows

Therefore:

Requiring a longer napkin for greater protection: the back portion of the napkin may be higher/longer in length or equal in length with the front. It can vary dependent upon the need* *Keep in mind the product size will also vary in order to accommodate the size of the wearer: small, medium, large or ex-large according to the style of the panti.

The medium is for those days in between when the flow is moderate so its' length may or may not be as long.

Again, dependent upon the flow and varying in length.* *Keep in mind the product size will also vary in order to accommodate the size of the wearer: small, medium, large or ex-large according to the style of the panti.

The lite is for those days, when the monthly (period) is lite/to stop and therefore only requiring a lighter protection so the pad will not need to be as long: inother words, for those spotting days. Varying in length* *Keep in mind the product size will also vary in order to accommodate the size of the wearer: small, medium, large or ex-large according to the style of the panti.

Note: The thickness can and may vary, or basically stays the same. Again, dependent upon the type of protection needed: thicker in front, back, both ends or just basically the regular contoured style.

Size/Style:

The overall product is a panti shaped product that varies in sizes, that can come in full panti size brief size or bikini size or thong and varies sizewise. It can come in small, medium, large or ex-large. Varying in sizes with a limit applicable to all.

The panti comes in white, beige (fleshtone) and black and the sanitary napkin will be white. Options: beige (fleshtone) or black.

Note: The crouch area of the panti will have the outer portion the color of the panti with the napkin sewn into the crouch area. (Options are open for the sanitary napkin being colored coordinated to the color of the panti).

The split visible in all three drawings indicate the wedge shape as part of the napkin that is sewn into the crouch area and varies (contour wise) according to the length of the napkin and adds to a close fit and gives room (expandability) of length for the flow.

The leg areas: front and back outer trim is elasticized for snug panty-like fit.

The pad will expand beyond the crouch area: branching forward, backward, longer, shorter, equal or unequal in distance.

Note the overview design showing a branching or broadening in the napkin.

Note: the use of the panti wording versus panty is as a referral to my patent and I would like for this word to be patented if not will try copyrighting.

1. What I claim as my invention is a unique product that addresses the sanitary needs of women/young girls by providing a product that is manufactured as an underwear and sanitary product and comes in many forms of underwear protection:
The uniqueness of this invention is also in that the sanitary napkin and underwear garment is a disposable, “all in one” product.
This invention is manufactured in varying panti (underwear) styles: full, brief and thong, etc.

2. The sanitary napkin portion of this invention is interwoven or sewn in the crouch area and varies in length within and from the crouch area of the underwear:
It can be long in front and short in back, or short in back and long in front or it can be equally long, both front and back and equally, short both front and back. Again, varying according to needs and individual users.
